(19) United States
(12) Patent Application Publication
(10) Pub. No.: US 2018/0164224 A1
(43) Pub. Date: Jun. 14, 2018

(54) Apparatus for Photographing Glass in Multiple Layers

(71) Applicant: ASA Corporation, Rocklin, CA (US)

(72) Inventor: LIANSHENG JIANG, Rocklin, CA (US)

(21) Appl. No.: 15/377,718

(22) Filed: Dec. 13, 2016

(51) Int. Cl. G01N 21/89; G01N 21/896; G01N 33/38

(52) U.S. Cl. CPC G01N 21/8901; G01N 21/896; G01N 33/386; G01N 2021/845; G01N 2201/062; G01N 2201/101; G01N 21/8914

(57) ABSTRACT

The invention discloses a new apparatus to photograph glasses in multiple layers for taking high quality photo images with scratch, crash, black/white defect, lack, crack, pin-hole, concave edge and raised edge, bubble and smudge defects on the surface-layer, backside-layer or/and mid-layer of the glasses. The invention also introduces flexible and expendable photographing hardware architecture that will meet various customers inspecting defects requirements and speed requirements.

### CROSS REFERENCE TO RELATED APPLICATIONS

This application discloses and claims only subject matter disclosed in prior application Ser. No. 14/178,295 filed Feb. 12, 2014, and names the inventor who was named in the prior application. Accordingly, this application may constitute a continuation or divisional, and therefore claims the benefit of priority to the prior application Ser. No. 14/178,295 that is a continuation-in-part of the PCT application No. PCT/CN2012/074463 that further claimed the priority benefit to the U.S. Provisional Application No. 61/517,620.

### FIELD OF THE INVENTION

The present invention generally relates to the field of glass inspection technology. More particularly, this invention relates to the field of machine-vision applications, involves motion control, optical, photography design, and apparatus hardware architecture. Yet more particularly, the invention relates to a consistent, stable, fast, and high accuracy glass photographing apparatus for photographing different defects (such as scratches, crashes, black/white defects, lack, cracks, pin-holes, concave edges and raised edges, bubbles and smudges) on the surface, backside and mid-layer of glasses in high accuracy.

### BACKGROUND OF THE INVENTION

Glass inspection is applied in many fields, especially in the field of mobile phone display panel, iPad window panel and flat-panel display manufactures. Due to difficulty for taking high quality images on different defects (scratches, crashes, black/white defects, lack, cracks, pin-holes, concave edges and raised edges, bubbles and smudges) on the surface, backside and mid-layer of glasses, therefore in the world, no full automatic glass inspection system provides high quality inspection solution, thus the method of human manual inspections is still used in most glass manufactures. The inspection accuracy of this method is limited by the vision of human beings and the inspection results of this method can become not reliable due to human errors from human's feeling, emotion and tiredness.

Among the current automatic glass detection technology, there are some apparatus that can photograph one or several defects, but it still lacks a consistent, stable, fast, and high accuracy glass photographing apparatus, and the method for creating the same.

Among the current automatic glass detection technology, most of glass photography devices use multiple area scanning cameras for high accuracy photography, but this technique is restricted at its mechanical mounting space when photographing small size glasses such as mobile phone glass panel. This invention uses multiple line-scanning cameras with micro-lens to walk around the problem of space limit.

Among the multiple layer glass photography of the invention, multiple layer photography for scratches on glasses is most difficult. So far there is no single apparatus that can photograph glass scratches at all orientations. For instance, one apparatus is able to take clear photographs to expose scratches on glasses on vertical direction (+/−300) but not on horizon direction, and another apparatus is able to take clear scratch photographs on glasses on horizon direction (+/−300) but not on vertical direction. To solve the glass photograph problem that it is difficult to get clear photographs to expose scratches on all orientations, this invention introduces an apparatus that adopts an illumination technology using multiple lights to spread on the glass from different positions and various angles.

Most of machine-vision applications take one photograph of glass panel for image process and inspection. The apparatus of this invention introduces flexible hardware architectures. Using the apparatus of this invention, it can take one or multiple photo-images for each glass panel based on the glass inspection need of customer, and each photo-image can capture clear image of one or more defects in various layers. This results in fast inspections of multiple-layered glass with the support of multi-tasking image process and inspection.

On normal machine vision applications, the photographing hardware is usually fixed, and is not easy to modify. This invention introduces a flexible hardware architectures for various glass inspecting purposes.

There are two methods to take photo-image on glasses. One is fixing glasses and move cameras, and the other is fixing cameras and moving glasses. The later method is used in this invention.

To control photographing and real-time inspection at multiple glass layers, multi-tasking photographing, image processing and inspection techniques software must be programmed.

### SUMMARY OF THE INVENTION

This invention introduces a multiple camera process when each camera will take one full image for inspecting one or more defect(s) on multiple glass layer(s). It involves an apparatus for photographing defects (scratches, crashes, black/white defects, lack, cracks, pin-holes, concave edges and raised edges, bubbles and smudges) on multiple layers of glasses. The apparatus contains the following hardware components: a conveyor, one or more line-scan or area-scan camera(s), one or more line-light(s) or area-light(s), and one or more normal lens or micro-lens.

This invention introduces an apparatus to get clear photographs to expose scratches on a glass panel by using multiple lights that spread line beams from different positions and angles. The line-scan camera is mounted vertically with the glass panel, and two line-lights are mounted in parallel to the camera's scan-line and spread the first two line beams on the camera's scan-line, another two line-lights spread two line beams from both sides of camera, merging with the first two line beams on the camera's scan line. This technique is able to get clear photographs to expose scratches on all orientations.

This invention introduces an apparatus to get clear photographs to expose silk print defects on a glass panel. A line-scan camera is mounted on the topside of the glass panel and is vertical with the glass panel, one line-light is mounted in parallel to the camera's scan-line and spreads the line beam on the camera's scan-line on the surface of the glass panel. This technique is able to get clear photographs to expose silk print defects.

This invention introduces an apparatus to get clear photographs to expose black/white defects: A line-scan camera is mounted above the glass panel and is vertical to the glass panel, two line-lights are mounted above the glass panel and spread line beams on the camera's scan-line from above the glass panels. This technique is able to get clear photographs to expose black/white defects.

This invention introduces an apparatus to get clear photographs to expose side-crash or lack defects: A line-scan camera is mounted above the glass panel and is vertical to the glass panel, one line-light is mounted at backside of the glass panel and spreads a line beam on the camera's scan-line on the backside of the glass panel. This technique is able to get clear photographs to expose side-crash or lack defects.

This invention introduces an apparatus to get clear photographs to expose cracks defects: A line-scan camera is mounted at back side of the glass panel and is vertical to the glass panel, one line-light is mounted at backside of the glass panel and spreads a line beam on the camera's scan-line on the backside of the glass panel. This technique is able to get clear photographs to expose cracks defects.

This invention introduces an apparatus to get clear photographs to expose pin-hole defects: A line-scan camera is mounted above the glass panel and is vertical to the glass panel, two line-lights are mounted at backside of a glass panel and spread line beams on the camera's scan-line on the backside of the glass panel. This technique is able to get clear photographs to expose pin-hole defects.

This invention introduces an apparatus to get clear photographs of concave edges and raised edges to expose defects: A line-scan camera is mounted above the glass panel and is vertical to the glass panel, one line-light is mounted at backside of the glass panel and spreads a line beam on the camera's scan-line on the backside of the glass panel. This technique is able to get clear photographs of concave edges and raised edges to expose defects.

This invention introduces an apparatus to get clear photographs of bubble and/or smudge to expose defects: A line-scan camera is mounted above the glass panel and is vertical to the glass panel, two line-lights are mounted on the topside and on the backside of the glass panel respectively and spread line beams on the camera's scan-line. This technique is able to get clear photographs of bubble and/or smudge to expose defects.

This invention introduces flexible hardware architectures for various glass inspecting purposes. Depending on total types of inspecting glass defects from the individual customer required, various hardware systems can be designed by mounting multiple cameras and line-lights, and each camera will photograph one or more defects on the glass. In such kind of customized hardware system, all spots of glass defects that are required to inspect by a customer will be photographed.

### BRIEF DESCRIPTION OF THE DRAWINGS

All schematic diagrams included in this invention do not contain distances between the cameras and the glass panels since the distances depend on (1) Resolution of line-scan camera, for instance IK, 2K, 4K, 8K, 12K or 16K line-scan cameras or larger scale area-scan cameras are popular in machine vision applications, (2) Width of glasses, and (3) Defects inspection accuracy requirements. Any selection of them will result in varies of distances between the cameras and the glass panels.

FIG. 1-1 is a schematic diagram of the invention for exposing scratches on the surface or backside of glasses.

FIG. 1-2 is a schematic diagram illustrating the conjunction angle between camera's scan-line and line-lights according to FIG. 1-1.

FIG. 1-3 is a schematic illustrating the merged light beams that spread on the camera's scan line according to FIG. 1-1.

FIG. 2-1 is a schematic diagram of the invention for exposing silk print defects on the glasses.

FIG. 2-2 is a schematic diagram illustrating the conjunction angle between camera's scan-line and the line-light according to FIG. 2-1.

FIG. 2-3 is a schematic diagram of FIG. 2-1 illustrating the light beam that spreads on the camera's scan-line according to FIG. 2-1.

FIG. 3-1 is a schematic diagram of the invention for exposing black/white defects on the glasses.

FIG. 3-2 is a schematic diagram illustrating the conjunction angle between camera's scan-line and line-lights according to FIG. 3-1.

FIG. 3-3 is a schematic diagram showing the light beams that spread on the camera's scan-line according to FIG. 3-1.

FIG. 4-1 is a schematic diagram of the invention for exposing side-crash and lack defects on the glasses.

FIG. 4-2 is a schematic diagram illustrating the conjunction angle between camera's scan-line and the line-light according to FIG. 4-1.

FIG. 4-3 is a schematic diagram illustrating the light beam that spreads on the camera's scan-line according to FIG. 4-1.

FIG. 5-1 is a schematic diagram of the invention for exposing cracks on the glasses.

FIG. 5-2 is a schematic diagram illustrating the conjunction angle between camera's scan-line and the line-light according to FIG. 5-1.

FIG. 5-3 is additional schematic diagram of FIG. 5-1 to show the light beam that spreads on the camera's scan-line according to FIG. 5-1.

FIG. 6-1 is a schematic diagram of the current invention for exposing pin-holes on the glasses.

FIG. 6-2 is a schematic diagram illustrating the conjunction angle between camera's scan-line and line-lights according to FIG. 6-1.

FIG. 6-3 is a schematic diagram illustrating the light beams that spread on the camera's scan-line according to FIG. 6-1.

FIG. 7-1 is a schematic diagram of the invention for exposing concave edges and raised edges on the glasses.

FIG. 7-2 is a schematic diagram illustrating the conjunction angle between camera's scan-line and the line-light according to FIG. 7-1.

FIG. 7-3 is a schematic diagram illustrating the light beam that spreads on the camera's scan-line according to FIG. 7-1.

FIG. 8-1 is a schematic diagram of the invention for exposing bubbles and smudges on the glasses.

FIG. 8-2 is a schematic diagram illustrating the conjunction angle between camera's scan-line and line-lights according to FIG. 8-1.

FIG. 8-3 is a schematic diagram illustrating the lighting beams that spread on the camera's scan-line according to FIG. 8-1.

FIG. 9 is a sample device hardware architecture schematic diagram, which photographs the mobile phone glasses before the silk printing.

FIG. 10 is a sample device hardware architecture schematic diagram, which photographs the mobile phone glasses after the silk printing.

### DESCRIPTION OF THE INVENTION

This invention introduces an apparatus for photographing the defects including but not limit to scratches, cracks, concave and raised edges, bubbles and smudges on the surface, backside and mid-layer of glass(es). The mechanism of the apparatus associated with photography involves a conveyor, one or more line-scan or area-scan camera(s), one or more line-light(s) or area-light(s), and one or more normal lens (for low accuracy) or micro-lens (for high accuracy) depending on accuracy of inspection requirements. The conveyor can be roller conveyor, air floating conveyor or any other type of conveyor; the selected conveyor for line-scan camera must leave enough gaps for line-scanning; the light source is a strip-shaped light source made of LED lights or other type of lights, including strip-shaped line-light source or strip-shaped area-light source; the lens is the normal lens or micro-lens; and the computer is for devices (conveyor, camera, lighting source) controls and for glass image acquisitions. Since the techniques of conveyor, camera and lens is beyond this invention, they will not be described more in details.

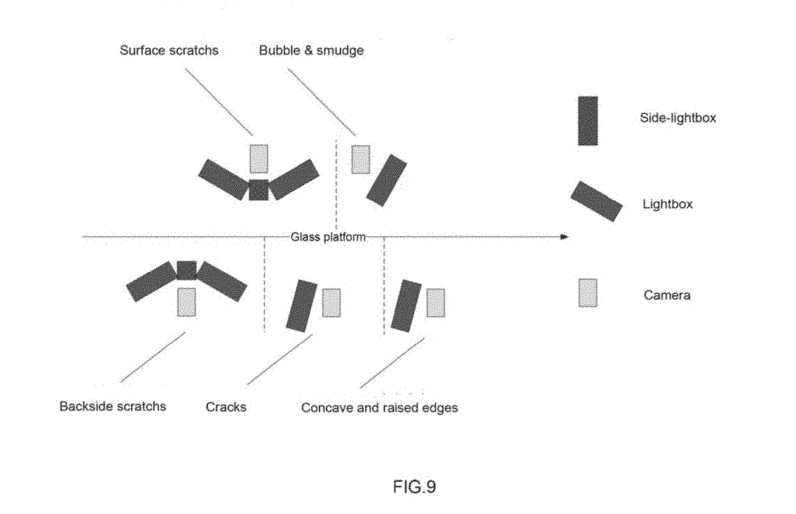

This invention also introduces flexible and expendable photographing hardware architectures according to customer inspecting defects requirements and speed requirements. For instance, to inspect the mobile phone glasses before silk-printing, the requested photographing spots of defects include scratches, cracks, concave and raised edges, bubbles and smudges on the surface, backside and mid-layer of glasses. To construct such kind of inspection system, we set two line-scan cameras and six line-lights for exposing scratches on surface and backside of glasses, another three line-scan cameras and three line-lights shall be involved for exposing cracks, concave and raised edges, bubbles and smudges. FIG. 9 is a schematic diagram illustrating a sample hardware configuration for exposing mobile phone glass before silk printing. The iPad front panel glass inspection shall be constructed the same way for photography.

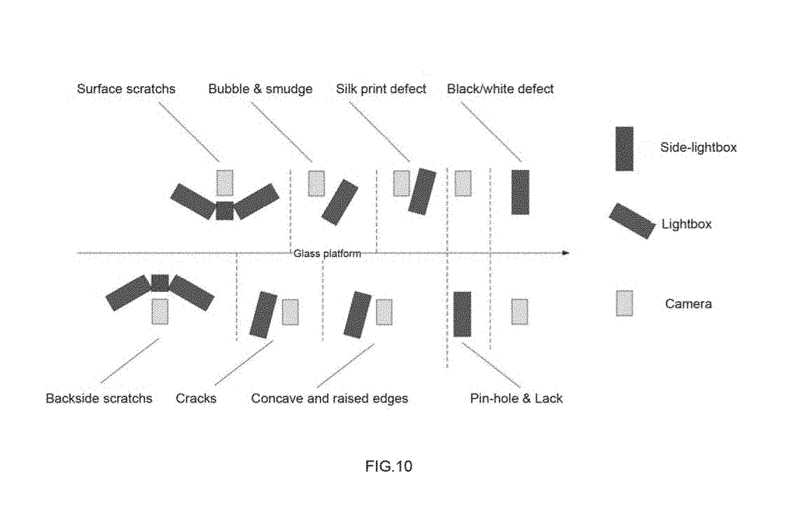

As another instance, after silk-printing, the mobile phone glass maker will request to photograph spots to expose defects include scratches, silk print defect, black/white defects, lack, cracks, pin-holes, concave and raised edges, bubbles and smudges on the surface, backside and mid-layer of glasses. This comprehensive inspection will request up to eight line-scan cameras and twelve line-lights. Mainly more cameras and line-lights will raise the productivities of the glass inspection. FIG. 10 is a schematic diagram illustrating a sample hardware configuration for inspecting mobile phone glass after silk printing.

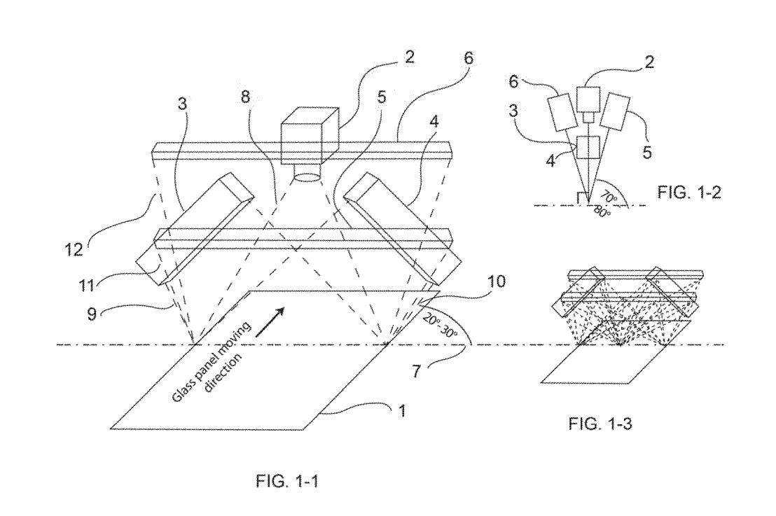

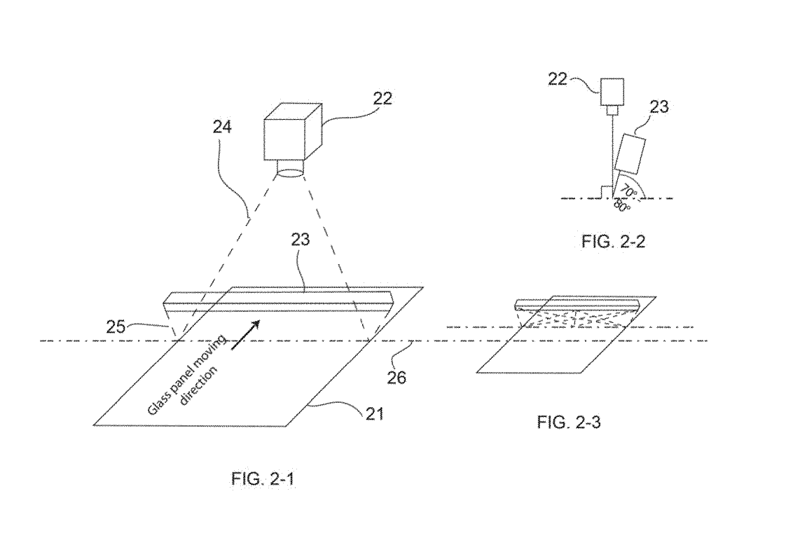

This invention introduces an apparatus to get clear scratch(es) photographs using multiple lights spread from different positions and angles. FIG. 1-1 is a schematic diagram illustrating the hardware configuration for scratch inspection according to the present invention, and FIG. 1-2 is a schematic diagram illustrating the camera-light angle is 70°˜80° according to FIG. 1-1. The line-lights (5 & 6) spread the first two line beams on the camera's scan-line (7), another two line-lights (3 & 4) spread two line beams (9 & 10) from both sides of the camera (2), the angle of line beams (9 & 10) and camera's scan-line (7) is about 20°˜30° for exposing the scratches in +/−30° of orientation from glass panel moving direction. This structure of line-lights (3, 4, 5 & 6) results in spreading and merging the line beams (9, 10, 11 & 12) on the entire camera's scan-line (7). FIG. 1-3 is an optical path diagram illustrating how three points (left-edge-point, mid-point and right-edge-point) on camera's scan line (7) are illuminated by lights in many directions from line-lights (3, 4, 5 & 6) as shown in FIG. 1-1. It is clear that any point on camera's scan line (7) will be spread by lights from various directions from the line-lights (3, 4, 5 & 6), it will guarantee all the scratches (in any orientation) be exposed when the line-scan camera (2) photographs line by line and make a clear scratch picture. Without line-lights (3 and/or 4) in FIG. 1, it is not able to expose scratches in +/−30° of orientation from glass panel moving direction; and without line-lights (5 & 6) in FIG. 1, it is not able to expose scratches in +/−30° of orientation from camera's scan-line (7). In FIG. 1-2, why the camera and light-beam angle is about 70°˜80° ? Because keeping the line-light (5 & 6) enough vertically will make the line beams (11 & 12) spreading deeply into scratches for exposing deeper scratches.

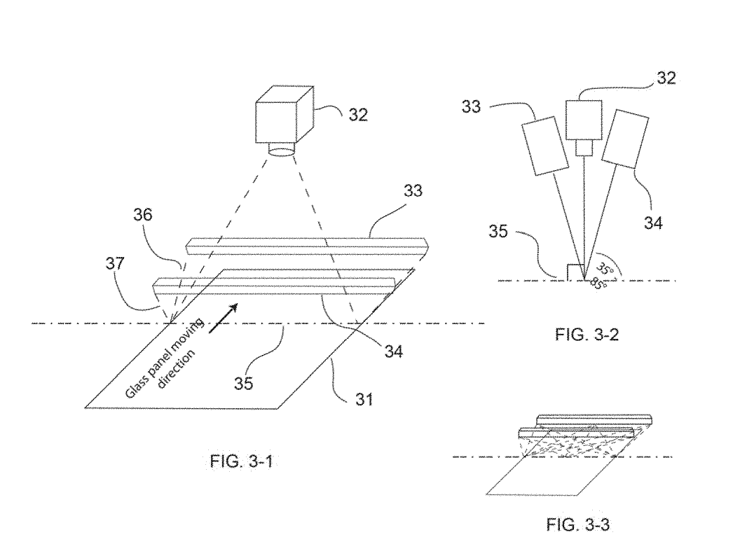

This invention introduces an apparatus to get clear silk print defect photographs using one line-light. FIG. 2-1 is a schematic diagram illustrating the hardware configuration for silk print defect inspection apparatus according to the present invention. FIG. 2-2 is a schematic diagram illustrating the camera-light angle according to FIG. 2-1. FIG. 2-3 is an optical path diagram illustrating how three points (left-edge-point, mid-point and right-edge-point) on camera's scan-line (26) are illuminated by lights in many directions from line-light (23) as shown in FIG. 2-1. It is clear that any point on camera's scan-line (26) will be spread by lights from various directions from the line-lights (23), it will guarantee all the silk print defects (in any orientation) be exposed when the line-scan camera (22) photographs line by line and make a clear silk print defect picture. In FIG. 2-2, why the camera and light-beam angle is about 70°˜80°. It is because keeping the line-light (23) enough vertically will make the line beam (25) spreading deeply since the silk printing material is thicker relatively in micro-photographing.

This invention introduces an apparatus to get clear black/white defect photographs using two line-lights. FIG. 3-1 is a schematic diagram illustrating the hardware configuration for black/white defects inspection apparatus to the present invention; and FIG. 3-2 is a schematic diagram illustrating angle between the glass and the light beams is 35°˜85° according to FIG. 3-1. The line-scan camera (32) is mounted on the top of glass panel (31) vertically with the glass panel (31), and two line-lights (33 & 34) are mounted on the top side of glass panel in parallel with camera's scan-line (35), and spread light beams (36 & 37) on the camera's scan-line (35). FIG. 3-3 is an optical path diagram illustrating how three points (left-edge-point, mid-point and right-edge-point) on camera's scan-line (35) are illuminated by lights in many directions from line-light (33) as shown in FIG. 3-1. It is clear that any point on camera's scan-line (35) will be spread by lights from various directions from the line-lights (33 & 34), when the light beams (36 & 37) pass through the glass panel (31), the black/white defects will block the part of light beams (36 & 37), different defect's color (black or white) or layer will result in various shape and gray-scale on the photograph. Therefore, all the black/white defects will pass through the light beams (36 & 37) and be exposed. And the line-scan camera (32) photographs line by line and makes a clear black/white defects picture.

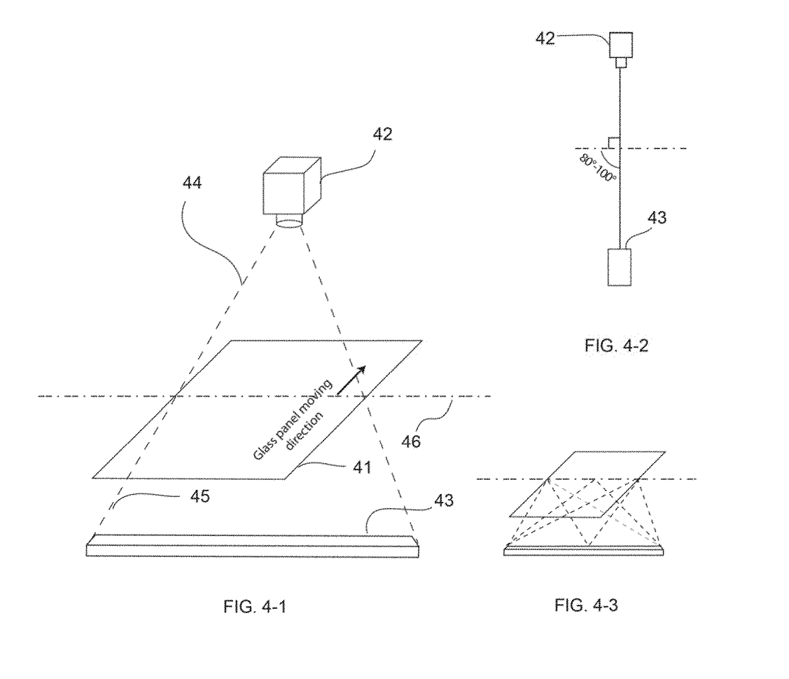

This invention introduces an apparatus to get clear side-crash and lacks photographs using one line-light. FIG. 4-1 is a schematic diagram illustrating the hardware configuration for lacks inspection apparatus according to the present invention and FIG. 4-2 is a schematic diagram illustrating the angle between the glass and the light beam is 80°˜100° according to FIG. 4-1. The line-scan camera (42) is mounted on the top of glass panel (41) vertically with the glass panel (41), and one line-light (43) is mounted on the backside of glass panel in parallel with camera's scan-line (46), and spreads light beam (45) with the camera's scan-line (46). FIG. 4-3 is an optical path diagram illustrating how three points (left-edge-point, mid-point and right-edge-point) on camera's scan-line (46) are illuminated by lights in many directions from line-light (43) as shown in FIG. 4-1. It is clear that any point on camera's scan-line (46) will be spread by lights from various directions from the line-light (43). When the light beam (45) pass through the glass panel (41), it will also pass through the lacks and lacks will be exposed. And the line-scan camera (42) photographs line by line and makes a clear side-crash and lacks picture.

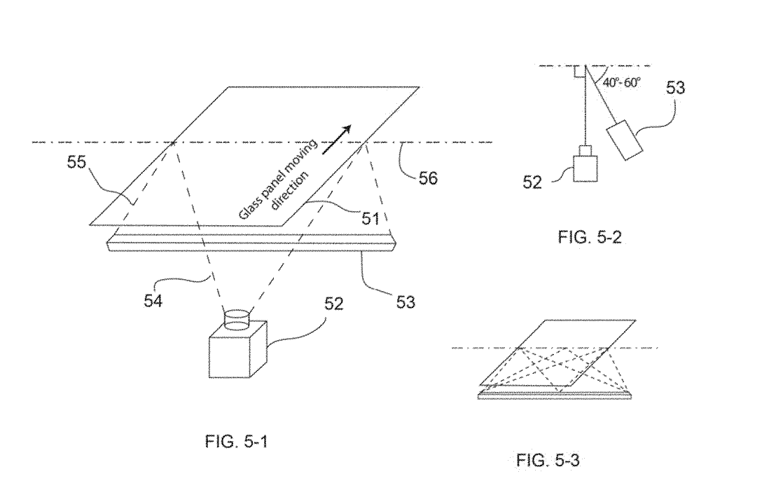

This invention introduces an apparatus to get clear crack photographs using one line-light. FIG. 5-1 is a schematic diagram illustrating the hardware configuration for crack inspection apparatus according to the present invention and FIG. 5-2 is a schematic diagram illustrating the angle between the glass and the light-beam is 40°˜60° according to FIG. 5-1. The line-scan camera (52) is mounted on the backside of glass panel (51) vertically with the glass panel (51), and one line-light (53) is mounted on the backside of glass panel in parallel with camera's scan line (56), and spreads light beam (55) merging with the camera's scan-line (56). FIG. 5-3 is an optical path diagram illustrating how three points (left-edge-point, mid-point and right-edge-point) on camera's scan-line (56) are illuminated by lights in many directions from line-light (53) as shown in FIG. 5-1. It is clear that any point on camera's scan line (56) will be spread by lights from various directions from the line-light (53). When the light beam (55) pass through the glass panel (51), will also pass through the cracks and crack's edges will be exposed clearly. And the line-scan camera (52) photographs line by line and makes a clear crack picture.

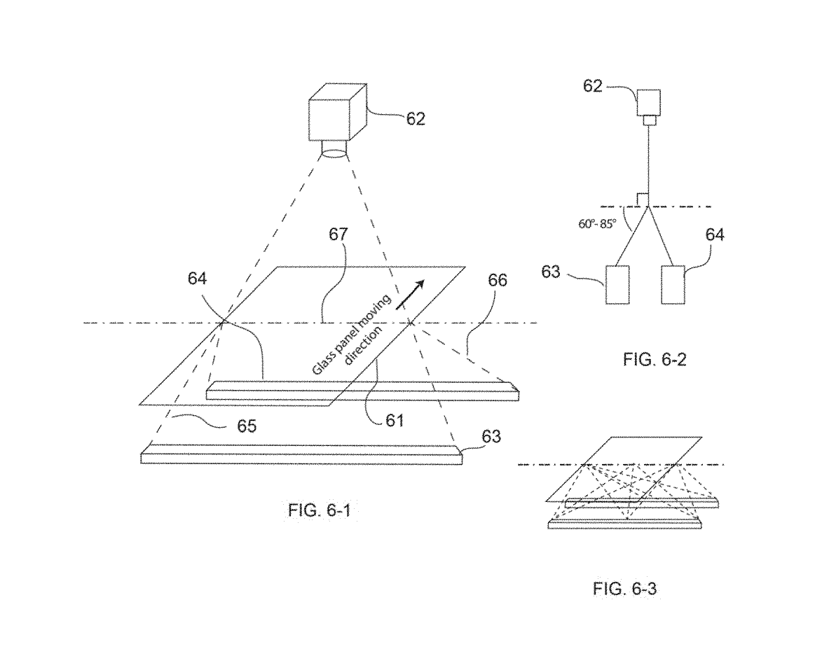

This invention introduces an apparatus to get clear pin-hole photographs using two line-lights. FIG. 6-1 is a schematic diagram illustrating the hardware configuration for pin-hole inspection apparatus according to the present invention and FIG. 6-2 is a schematic diagram illustrating the angle between the glass and the light-beams is 60°˜85° according to FIG. 6-1. The line-scan camera (62) is mounted on the top of glass panel (61) vertically with the glass panel (61), and two line-lights (63 & 64) are mounted on the backside of glass panel in parallel with camera's scan line (67), and spreads light beams (65 & 66) with the camera's scan-line (67). FIG. 6-3 is an optical path diagram illustrating how three points (left-edge-point, mid-point and right-edge-point) on camera's scan-line (67) are illuminated by lights in many directions from line-lights (63 & 64) as shown in FIG. 6-1. It is clear that any point on camera's scan line (67) will be spread by lights from various directions from the line-lights (63 & 64). When the light beams (65 & 66) pass through the glass panel (61), they will also pass through the pin-hole and pin-hole will be exposed. Since some pin-holes are tiny, it is not bright enough to use only one line-light and two line-lights are used. And the line-scan camera (62) photographs line by line and makes a clear pin-hole picture.

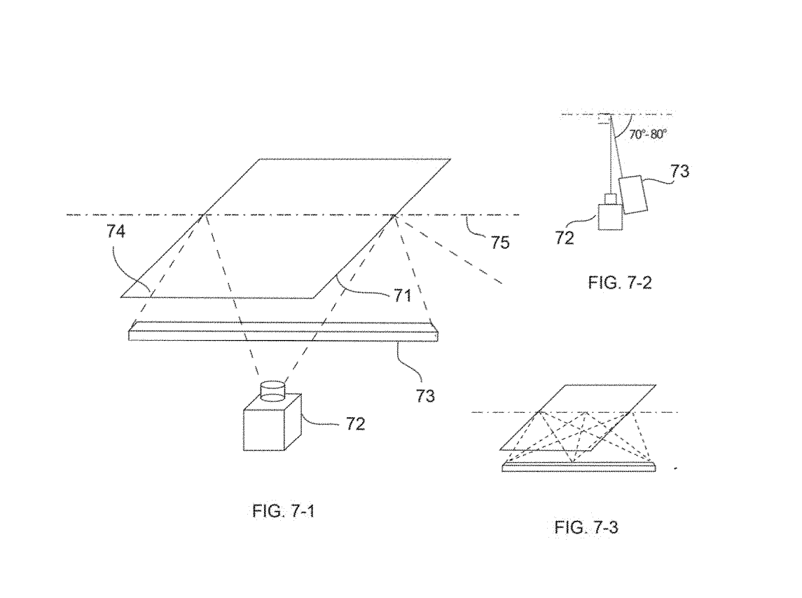

This invention introduces an apparatus to get clear concave and raised edge photographs using one light. FIG. 7-1 is a schematic diagram illustrating the hardware configuration for concave and raised edge inspection apparatus according to the present invention and FIG. 7-2 is a schematic diagram illustrating the angle between the glass and the light-beam is 70°˜80° according to FIG. 7-1. The line-scan camera (72) is mounted on the backside of glass panel (71) vertically with the glass panel (71), and one line-light (73) is mounted on the backside of glass panel (71) in parallel with camera's scan line (75), and spreads light beam (74) merging with the camera's scan-line (75). FIG. 7-3 is an optical path diagram illustrating how three points (left-edge-point, mid-point and right-edge-point) on camera's scan-line (75) are illuminated by lights in many directions from line-light (73) as shown in FIG. 7-1. It is clear that any point on camera's scan-line (75) will be spread by lights from various directions from the line-light (73), and concave and raised edge will be exposed clearly. And the line-scan camera (72) photographs line by line and makes a clear concave and raised edge.

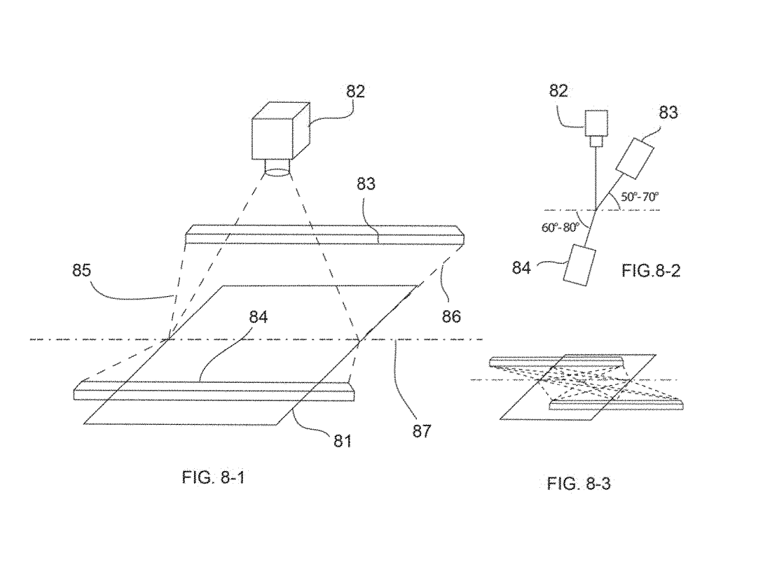

This invention introduces an apparatus to get clear bubble and smudge photographs using two line-lights. FIG. 8-1 is a schematic diagram illustrating the hardware configuration for bubble and smudge inspection apparatus according to the present invention; and FIG. 8-2 is a schematic diagram illustrating the angle between the glass panel and the topside and backside light beams is 50°˜70° and 60°˜80° respectively according to FIG. 8-1. FIG. 8-3 is an optical path diagram illustrating how three points (left-edge-point, mid-point and right-edge-point) on camera's scan-line (87) are illuminated by lights in many directions from line-lights (83 & 84) as shown in FIG. 8-1. The camera (82) is mounted at the top of the glass panel (81) vertically with the glass panel (81) and the two line-lights (83 & 84) are mounted at the top and back side of the glass panel respectively and spread line beams (85 & 86) which merge at the camera's scan line (87). It is clear that any point on camera's scan line (87) will be spread by lights from various directions from the line-lights (83 & 84), thus it will guarantee all the bubble and smudge defects (in any orientation) be exposed, when the line-scan camera (82) photographs line by line and make a clear bubble and smudge defect picture.

Illustrated in FIG. 9 is a schematic diagram illustrating a sample device hardware architecture that photographs the mobile phone glasses before the silk printing. FIG. 10 is a schematic diagram illustrating a sample device hardware architecture that photographs the mobile phone glasses after the silk printing.

Although one or more embodiments of the newly improved invention have been described in detail, one of ordinary skill in the art will appreciate the modifications to the material selection and the addition of a secondary dual screen option along with the new footprint layout of the laptop device. In particular, by adding the secondary dual screen option to laptop technology, users can perform tasks that are currently tedious to accomplish with only one main screen. It is acknowledged that obvious modifications will ensue to a person skilled in the art. The claims which follow will set out the full scope of the claims.

1: An apparatus for photographing glass(es) to expose defects, comprising: at least one camera, at least one light source, at least one computer and/or a conveyor;
wherein said defects comprising of one or more of the following types: scratches, silk print defects, black/white defects, lacks, cracks, pin-holes, concave and raised edges, bubbles and smudges on the surface, backside or/and mid-layer of the glass(es);
wherein the angle and distance between said glass and said light sources is designed based on the type of said defects;
wherein the angle and distance between said glass(es) and at least one cameras is designed based on the type of said defects;
wherein said camera is a line-scan camera or area-scan camera; and
wherein said light source is a strip-shaped light source made of LED lights or other type of lights.

2: The apparatus for photographing glass(es) of claim 1, said camera is mounted on the topside of said glass(es) or on the bottom of backside of said glasses; the distance between said camera and said glass(es) is between 50-1500 mm; one or more strip-shaped light sources are mounted at any position surrounding said camera, and every light source spreads over the entire scanning area of said camera; the length of said strip-shaped light sources is greater than the width of said glass(es); and the width of said strip-shaped light sources is between 2 mm and the length of said glass(es).

3: The apparatus for photographing glass(es) to expose defects of claim 1, wherein in exposing silk print defects, said camera is mounted vertically with said glass(es); wherein one line light is mounted at the same side of said camera and spreads a line beam at said camera's scan-line; wherein the angle between said light beam and said camera's scan-line is 70°˜80°; and wherein said apparatus is able to take clear photographs for exposing said silk print defects on said glass(es).

4: The apparatus for photographing glass(es) to expose defects of claim 1, wherein in exposing black/white defects photographs, said camera is mounted vertically on the topside of said glass(es), and two line-lights are mounted on the topside of said glass(es) and spread two line beams on said camera's scan-line; wherein the angle between said light beam and said camera's scan-line is 35°˜85°; and wherein said apparatus is able to take clear photographs for exposing said black/white defects on said glass(es).

5: The apparatus for photographing glass(es) to expose defects of claim 1, wherein in exposing side-crash or lack defects of said glass(es), said camera is mounted vertically with said glass(es); wherein one line-light is mounted at the backside of said glass(es) and spreads a line beam on said camera's scan-line on said backside of said glass(es); wherein the angle between said light beam and said camera's scan-line is 80°˜100°; and wherein said apparatus is able to take clear photographs for exposing said side-crash or lash defects on said glass(es).

6: The apparatus for photographing glass(es) to expose defects of claim 1, wherein in exposing crack defects of said glasses, said camera is mounted vertically at backside of said glass(es); wherein one line-light is mounted at the backside of said glass(es) and spreads a line beam on said camera's scan-line; wherein the angle between said light beam and said camera's scan-line is about 40°˜60°; and wherein said apparatus is able to take clear photographs for exposing said crack defects on said glass(es).

7: The apparatus for photographing glass(es) to expose defects of claim 1, wherein in exposing pin-hole defects of said glass(es), said camera is mounted vertically at the topside of said glass(es); wherein two line-lights are mounted at the backside of said glass(es) and spread two line beams on said camera's scan-line; the angle between said two light beams and said camera's scan-line is about 60°˜85°; and said apparatus is able to take clear photographs for exposing said pin-hole defects on said glass(es).

8: The apparatus for photographing glass(es) to expose defects of claim 1, wherein in exposing concave edges and raised edges defects of said glass(es), said camera is mounted vertically at the backside of said glass(es); wherein one line-light is mounted at the backside of said glass(es) and spreads a line beam on said camera's scan-line; the angle between said light beam and said camera's scan-line is 70°˜80°; and wherein said apparatus is able to take clear photographs for exposing said concave edges and raised edges defects on said glass(es).

9: The apparatus for photographing glass(es) to expose defects of claim 1, wherein in exposing bubble and smudge defects of said glass(es), said camera is mounted vertically at the topside of glass(es); wherein two line-lights are mounted at the topside and the backside of said glass(es) respectively and spread two line beams on said camera's scan-line; wherein the angel between said line beam from said topside of said glass(es) and said camera's scan-line is 50°˜70°; wherein the angel between said line beam from said backside of said glass(es) and said camera's scan-line is 70°˜80°; and wherein said apparatus is able to take clear photographs for exposing said bubble and smudge defects on said glass(es).
